(19) United States
(12) Patent Application Publication
(10) Pub. No.: US 2006/0257334 A1
(43) Pub. Date: Nov. 16, 2006

(54) Mssn dispersion and method for producing the same

(75) Inventors: Gerd Dahms, Duisburg (DE); Holger Seidel, Duisburg (DE)

Correspondence Address: BIRCH STEWART KOLASCH & BIRCH, PO BOX 747, FALLS CHURCH, VA, 22040-0747, US

(73) Assignee: IFAC GmbH & Co.KG Institut fur Angewandte Colloidtechnologie, Duisburg (DE)

(21) Appl. No.: 10/550,193

(22) PCT Filed: Feb. 19, 2004
(86) PCT No.: PCT/EP04/01589

§ 371 Date: Jul. 10, 2006

(30) Foreign Application Priority Data
Mar. 21, 2003 (DE) 103 12 763.1

(51) Int. Cl. A61K 8/18

(52) U.S. Cl. 424/59; 977/926

(57) ABSTRACT

Membrane-structured solid nanoparticles having an average diameter in the range from 10 to 10 000 nm which are solid at 25° C. and have a combination of active compound vehicle particles and emulsifiers such as to form membranes which infiltrate the entire nanoparticles so that there are emulsifiers in the interior and on the surface of the nanoparticles are described. An aqueous vehicle dispersion in which there are solid active compound vehicle particles which are based on wax, polymer or lipid, have an average diameter in the range from 10 to 10 000 nm, and comprise at least one active pharmaceutical, cosmetic and/or food technology compound is produced by a) mixing the active compound with the wax-, polymer- or lipid-based active compound vehicle and at least one emulsifier which leads in stage b) to the formation of a lyotropic liquid-crystalline mixed phase, at a temperature above the melting or softening point of the active compound vehicle, to form a phase B, b) mechanically mixing the phase B with an aqueous phase A, which may comprise an emulsifier, at a temperature above the melting or softening point of the active compound vehicle, the weight ratio of phase B to phase A being 1:5 to 5:1, without high-pressure homogenization, to form a lyotropic liquid-crystalline mixed phase, c) diluting the mixed phase with an aqueous phase, which may comprise an emulsifier, at an aqueous-phase temperature which is below the melting or softening point of the active compound vehicle, with stirring and without high-pressure homogenization, to a desired final concentration of the dispersion.

The invention relates to a method for producing an aqueous active compound vehicle dispersion, to a dispersion of this kind and to drugs, cosmetics or food additives comprising it. The active compound vehicle comprises membrane-structured solid nanoparticles (MSSN).

Active pharmaceutical, cosmetic and/or food technology compounds are frequently encapsulated in active compound vehicles in order to obtain targeted release of the active compound or to protect it against chemical decomposition. The active compound vehicle may be adapted to the particular application and allows appropriate metering and release of the active compound. In the past, solid lipid nanoparticles, denoted SLN, were developed. They represent an alternative carrier system to emulsions and liposomes. The nanoparticles may comprise active hydrophilic or hydrophobic pharmaceutical compounds and may be administered orally or parenterally. Usually in this case nanoparticles having an average diameter in the range from 50 nm to 1 μm are used. A matrix material used, in contrast to the known emulsions, is a solid lipid. In order to ensure high bioacceptance and good in vivo breakdown, predominantly, physiologically compatible lipids or lipids comprising physiological components such as glycerides from endogenous fatty acids are used. In the course of production, it is usual to use emulsifiers or surfactants. Production takes place by means of high-pressure homogenization. In that case the lipid matrix used is melted and an active pharmaceutical compound is dissolved or dispersed in the melt. The active compound melt is usually dispersed with an aqueous surfactant solution at the same temperature and with stirring. The dispersion thus obtained is subsequently homogenized in the hot state in a high-pressure homogenizer, a plunger/slot homogenizer for example, at pressures in the range from 200 to 1500 bar. This produces an emulsion whose lipid phase, on cooling, recrystallizes into solid lipid nanoparticles.

An alternative possibility is to carry out cold homogenization, in which case the active pharmaceutical compound is again introduced into a melted lipid phase. The resulting mixed phase is subsequently cooled, and the solid is ground to a grain size in the range from 50 to 100 μm. The lipid particles thus obtained are subsequently dispersed in a cold surfactant solution, and the resulting dispersion is then subjected to high-pressure homogenization.

One method for producing SLN dispersions is described for example in EP-B-0 167 825. The lipid nanopellets described therein are used as a vehicle system for drugs intended for peroral administration. The lipid nanopellets are produced by dispersing the melted lipid with water, using a high-speed agitator. The desired particle size distribution is subsequently set by means of an ultrasound treatment. Stirring takes place generally at speeds in the region of 20 000 min⁻¹. The particles obtained have average diameters in the range from 100 to 1000 nm.

EP-B 0 605 497 describes drug vehicles comprising solid lipid particles (solid lipid nanospheres (SLN)). Production takes place by high-pressure homogenization or high-pressure dispersion at pressures of 500 to 1550 bar. The high-pressure homogenizer used is, for example, a slot homogenizer or a high-speed homogenizer. Preliminary dispersion is generally carried out using a rotor/stator disperser.

A similar method is described in U.S. Pat. No. 5,885,486. Colloidally distributed solid lipid particles are produced by high-pressure homogenization of a lipid melt with an aqueous phase. Again, operation takes place with pressures of 500 bar or more.

A review of the use of solid lipid nanoparticles as carriers for active pharmaceutical and cosmetic compounds is found in J. Microencapsulation, 1999, vol. 16, No. 6, pages 751 to 767. It includes in particular a description of how vitamin E is introduced into SLN systems. There is a description of how, as a result of introduction into solid lipid nanoparticles, improved penetration and action of the vitamin E on the skin is achieved.

J. Cosmet. Sci., 52, pages 313 to 324 describes the occlusion effects of solid lipid nanoparticles. A particular subject of investigation is the effect of skin moistening. An SLN formulation containing 40% cetyl palmitate and 5% surfactant in water was realized by means of high-speed agitators; see formulation CPe in table I. An average particle diameter of 3 μm was found; see table II.

The production of solid lipid nanoparticles with low average particle diameter in accordance with the prior art is costly and inconvenient, since it generally requires the use of high-pressure homogenizers. Simple stirring at high speed produces only relatively large average particle diameters of 3 μm. The load capacity of the lipid particles is limited, and their morphology cannot always be readily controlled. Specific surface modifications are difficult to bring about.

It is an object of the present invention to provide an innovative system of solid nanoparticles which in comparison to known nanoparticles have a higher loading capacity, allow a greater selection of active compound vehicles and surfactants, and can be present at high concentrations in dispersions, and also a method for producing a solid nanoparticle dispersion, which avoids the drawbacks of the known methods and is not costly or inconvenient to implement. The intention in particular is that small particle diameters should be obtained for a low mechanical mixing effort. The intention, moreover, is to provide innovative solid nanoparticle dispersions which—like the nanoparticles—in particular have a high loading capacity, permit a wide range of active compound vehicles and emulsifiers, and allow surface modifications.

This object is achieved in accordance with the invention by a method for producing an aqueous vehicle dispersion, comprising in particular membrane-structured solid nanoparticles, in which there are solid active compound vehicle particles which are based on wax, polymer or lipid, have an average diameter in the range from 10 to 10 000 nm, and comprise at least one active pharmaceutical, cosmetic and/or food technology compound, fragrance or flavor, by
a) mixing the active compound with the wax-, polymer- or lipid-based active compound vehicle and at least one emulsifier which leads in stage b) to the formation of a lyotropic liquid-crystalline mixed phase, at a temperature above the melting or softening point of the active compound vehicle, to form a phase B,
b) mechanically mixing the phase B with an aqueous phase A, which may comprise an emulsifier, at a temperature above the melting or softening point of the active compound vehicle, the weight ratio of phase B to phase A being 1:5 to 5:1, without high-pressure homogenization, to form a—preferably gellike—lyotropic liquid-crystalline mixed phase,
c) diluting the mixed phase with an aqueous phase, which may comprise an emulsifier, at an aqueous-phase temperature which is below the melting or softening point of the active compound vehicle, for example at least 5° C. below, preferably at least 15° C. below, with stirring and without high-pressure homogenization, to a desired final concentration of the dispersion.

It has been found in accordance with the invention that aqueous active compound vehicle dispersions in which there are solid, lipid-based active compound vehicle particles having an average diameter in the range from 10 to 1000 nm can be produced advantageously if a lipid melt is mixed with an aqueous phase that has been heated to the same temperature in a defined weight ratio of 1:5 to 5:1. Mixing can be achieved in this case by means of conventional, mechanical agitators which have the agitation performance of a household mixer (or household kitchen mixer). In laboratory operation, for example, it was possible to achieve sufficient agitation using a Braun® kitchen mixer having a mixing head in the form of a double-armed propeller with a total diameter of 50 mm. The mixing propeller was surrounded by a protection ring having a diameter of 63 mm. The maximum power of the kitchen mixer was 350 W. The model in question was the MR 550, type 4189.

The mechanical mixing in stage b) and the stirring in stage c) take place preferably with agitators which have a peripheral speed in the range from 1 to 20 m/s, more preferably 1 to 3 m/s.

The shearing action of the agitator corresponds preferably to the shearing action of a household kitchen mixer of the above-described standard commercial type.

By observing the proportions of phases A and B it is possible to achieve a very strong mixing action even with the input of low shearing energies.

Without being tied to any theory, the lyotropic liquid-crystalline microemulsion obtained when phase B is mixed with the aqueous phase A can be understood as being a system of two interpenetrating networks, so that the microemulsion displays one-phase behavior. The microemulsion has a low viscosity under shear.

The weight ratio of phase B to phase A in stage b) is preferably 1:2 to 2:1, more preferably 1:1.5 to 1.5:1.

The object is further achieved in accordance with the invention by means of membrane-structured solid nanoparticles having an average diameter in the range from 10 to 10 000 nm which are solid at 25° C. and have a combination of active compound vehicle particles and emulsifiers such as to form membranes which infiltrate the entire nanoparticles so that there are emulsifiers in the interior and on the surface of the nanoparticles.

Preferably there are essentially no regions without a membrane structure over the cross section of the nanoparticles. The membranes are preferably formed in a lyotropic liquid-crystalline mixed phase which in the presence of water is self-emulsifying.

In contradistinction to the known SLN, emulsifiers the nanoparticles of the invention are present in the interior of the particles. The entire particles are composed of a membrane or membranes, whereas in SLN a solid core of the active compound vehicle is surrounded by a layer of emulsifier. Consequently the nano-particles, essentially independently of the scale in which they are viewed, have a uniform construction comprising membrane structures. The membrane-structured solid nanoparticles (MSSN) can be produced in accordance with the invention by the method described above. As compared with the SLN they are distinguished by a membrane structuring interspersed throughout the particles. Consequently there is a substantially larger membrane surface area into which active compounds can be embedded. It is therefore possible in accordance with the invention to introduce large amounts of active pharmaceutical, cosmetic and/or food technology compounds into the membranes or into the nanoparticles. By way of example it is possible to introduce amounts of up to 70% by weight, preferably up to 60% by weight, based on the loaded nanoparticles. These active compounds are stored not only in the surface region of the nanoparticles in the membranes, but throughout the particles. This enables active compounds to be released in a highly targeted way, including release over a prolonged period of time. The nanoparticles or lipid particles all in all, therefore, constitute a membrane which infiltrates the entire particles. This mutual infiltration is characteristic of the MSSN of the invention.

The membrane structuring can be achieved by means of known liquid-crystalline systems, such as lamellar, hexagonal or cubic liquid-crystalline systems.

The liquid-crystalline mixed phase is usually anisotropic and hence turbid or opaque.

The membrane-structured or lyotropic liquid-crystalline mixed phase possesses in the presence of water, self-emulsifying properties; in other words, an emulsification process occurs spontaneously at the interface with water. Even at high levels of lipid loading, the membrane-structured or lyotropic liquid-crystalline mixed phase displays electrical conductivity. In the course of production by the method described above, a liquid-crystalline gel state is passed through before or during the dilution with water. The dispersions obtained in the production method are free-flowing within a wide weight range of the MSSN phase. For example, dispersions with up to 60% by weight of MSSN phase, based on the overall dispersion, are free-flowing. Hence it is possible to produce free-flowing dispersions with, for example, 40% to 60% by weight of MSSN phase.

The MSSN can be loaded with any of a very wide variety of active compounds, as elucidated in more detail below. The maximum achievable loading level depends, among other things, on the melting point of the substance being loaded (active compound). Provided the active compound enjoys high solubility in the active compound vehicle, high levels of loading can be achieved.

The MSSN of the invention have a multiplicity of merits. Active compounds can be released in a targeted and delayed way. In the course of production it is possible to control not only the particle size but also the release characteristics.

On application to the skin, the penetration of the active substance into the skin may be raised as a result of the “plaster effect”. In this case the skin is caused to swell, the pores open, and the active compound can be instilled. With the MSSN it is possible to reduce the transepidermal water loss.

The MSSN can be produced using a multiplicity of emulsifiers and/or surfactants. In principle it is possible for (virtually) all conventional surfactants to be employed, in some cases in appropriate combination.

A further possibility in accordance with the invention is to achieve surface modification in the MSSN with the aid of surfactants. Through concomitant use or subsequent application of anionic, cationic, amphoteric or further surfactants it is possible to tailor the loading ratios and surface structures of the active compound vehicle and so to optimize its adsorption characteristics.

In particular it is possible in accordance with the invention to use emulsifiers which are pharmaceutically acceptable and/or have received approval under food law.

The concentration of emulsifier can be controlled down to very low concentrations. By way of example it is possible, based on the active compound vehicle, to use not more than 5% by weight, more preferably not more than 3% by weight, of surfactant; depending on the field of application, the lower limit for the amount of surfactant is approximately 0.05% by weight.

The MSSN dispersions of the invention are stable on storage and even with a high nanoparticle concentration enjoy very good fluidity.

For the purpose of stabilizing or modifying the interfaces it is also possible in addition to use hydrocolloids.

The solid form of the particles and the inclusion of the active compounds within the particles protect the included active compounds against oxidative degradation, since the ingress of oxygen is greatly reduced.

The MSSN of the invention are able to interact under certain circumstances with membrane-active emulsion droplets, by means of mass transfer via the aqueous phase. This implies a reversal of the Ostwald ripening principle. This interaction can be utilized advantageously for the performance properties.

As compared with the SLN technology, the selection of surfactants and wax or lipid structures which can be used is greatly expanded. Moreover, surface modifications are possible. As already mentioned, the MSSN can be produced without great cost or inconvenience and have a high loading capacity. Irrespective of the active compound vehicle, the properties can be adapted to the particular requirements. Different active compounds, for example, can also be introduced into the active compound vehicle phase by an alcoholic solution or phase, an ethanolic solution or phase for example, and embedded in a targeted way.

Hydrophobic, amphiphilic, and hydrophilic active compounds can be embedded simultaneously in the MSSN of the invention, since the membrane structures have both hydrophilic and hydrophobic regions.

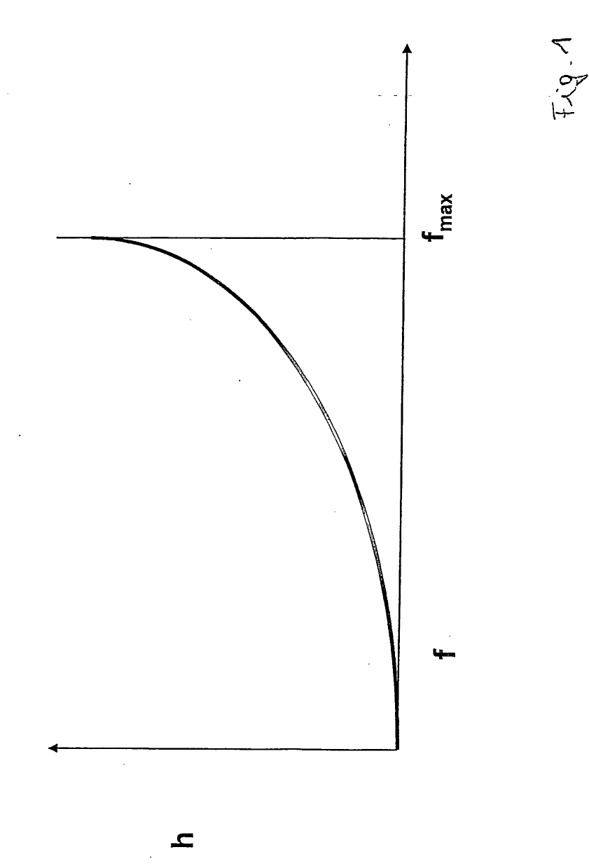

In the attached drawing FIG. 1 shows the relationship between the viscosity n and the volume f of the internal phase. Whereas the conventional production of emulsions operates far below the maximum internal phase volume f max in an emulsion, i.e., a 2- or 3-phase system, the invention operates slightly above this range, so that a mixed lyotropic liquid-crystalline phase is achieved.

Elucidated in more detail below are the active compound vehicles, suitable emulsifiers which form lamellar structures, suitable active pharmaceutical, cosmetic, and food technology compounds, and further possible ingredients of the aqueous active compound vehicle dispersion.

Active compound vehicle particles used are preferably lipid-based particles. They include lipids and lipid-like structures. Examples of suitable lipids are the di- and triglycerides of saturated straight-chain fatty acids having 12 to 30 carbon atoms, such as lauric acid, myristic acid, palmitic acid, stearic acid, arachidic acid, behenic acid, lignoceric acid, cerotic acid and melesinic acid, and their esters with other saturated fatty alcohols having 4 to 22, preferably 12 to 22 carbon atoms such as lauryl alcohol, myristyl alcohol, cetyl alcohol, stearyl alcohol, arachidyl alcohol, behenyl alcohol, saturated wax alcohols having 24 to 30 carbon atoms such as lignoceryl alcohol, cetyl alcohol, cetearyl alcohol and myristyl alcohol. Preference is given to di- and triglycerides, fatty alcohols, their esters or ethers, waxes, lipid peptides or mixtures thereof. Use is made in particular of synthetic di- and triglycerides as individual substances or in the form of a mixture, such as in the form of a hard fat, for example. Examples of glyceryl tri-fatty acid esters are glyceryl trilaurate, glyceryl trimyristate, glyceryl tripalmitate, glyceryl tri-stearate or glyceryl tribehenate. Waxes which can be used in accordance with the invention are natural waxes, such as plant waxes, animal waxes, mineral waxes and petrochemical waxes, chemically modified waxes, such as hard waxes, and synthetic waxes. For a listing of suitable waxes reference may be made to Römpp Chemielexikon, 9th edition, entry “Waxes”. Examples of suitable waxes are beeswax, carnauba wax, candelilla wax, paraffin waxes, isoparaffin waxes, and rice wax. Further examples of suitable waxes are cetyl palmitate and cera alba (bleached wax, DAB [German Pharmacopeia] 9). Suitable esters derive further, for example, from branched-chain fatty acids and fatty alcohols, glycerol, sorbitan, propylene glycol, methylglycoside, citric acid, tartaric acid, and mellitic acid. It is further possible to use ceramides, phytosphingosides, cholesterol, and phytosterols.

A further possibility is to use polymers such as silicone waxes and PVP derivatives. These are, for example, alkyl-substituted PVP derivatives, examples being tricontanyl-PVP, PVP-hexadecene copolymer, and PVP/eicosene copolymer. They can be used, for example, alone or as admixtures to the lipids as vehicle materials.

It is also possible to use liquid, semisolid and/or solid urethane derivatives, such as are sold, for example, by ALZO International Inc. These include, for example, fatty alcohol (branched) dimer/IPDI, fatty alcohol (linear) dimer/IPDI, ethoxylated fatty alcohol (branched) dmer/IPDI, ethoxylated fatty alcohol (linear) dimer/IPDI, dimethiconol/IPDI copolymers, triglyceride ester (hydrogenated)/IPDI copolymers, ethoxylated triglyceride ester (hydrogenated)/IPDI copolymers, aminated ethoxylated and non-ethoxylated triglyceride ester/IPDI copolymers.

The amount of active compound vehicle particles, based on the total aqueous active compound vehicle dispersion, is preferably 0.1% to 70% by weight, more preferably 1% to 60% by weight, for example, 0.1% to 30% or 1% to 10% by weight. In addition to the lipids it is possible to use dispersion stabilizers. They can be used, for example, in amounts of 0.01% to 20% by weight, preferably 0.05% to 5% by weight. Examples of suitable substances are surfactants, especially alkyl lactylates such as stearoyl lactylate, isethionates, alkyl sulfates such as sodium cetyl sulfate, diamide ether sulfates, alkylpolyglycosides, phosphoric esters such as sodium isotridecyl phosphate, taurates, sulfosuccinates, alkylpolyglycosides, alkyl sarcosinates such as sodium lauryl sarcosinate and alkyl glutamates such as sodium lauryl glutamate, ethoxylated sorbitan fatty acid esters, block polymers and block copolymers (such as poloxamers and poloxamines, for example), polyglycerol ethers and esters, lecithins of various origin (for example, egg lecithin or soybean lecithin), chemically modified lecithins (for example, hydrogenated lecithin), and also phospholipids and sphingolipids, mixtures of lecithins with phospholipids, sterols (for example, cholesterol and cholesterol derivatives and also stigmasterol), esters and ethers of sugars or sugar alcohols with fatty acids or fatty alcohols (for example, sucrose monostearate), sterically stabilizing substances such as poloxamers and poloxamines (polyoxyethylene-polyoxypropylene block polymers), ethoxylated sorbitan fatty acid esters, ethoxylated mono- and diglycerides, ethoxylated lipids and lipoids, ethoxylated fatty alcohols or fatty acids, and charge stabilizers or charge carriers such as, for example, dicetyl phosphate, phosphatidylglycerol, and saturated and unsaturated fatty acids, sodium cholate, sodium glycol cholate, sodium taurocholate or mixtures thereof, amino acids or peptizers such as sodium citrate (see J. S. Lucks, B. W. Müller, R. H. Müller, Int. J. Pharmaceutics 63, pages 183 to 18 (1990)), viscosity enhancers such as cellulose ethers and cellulose esters (for example, methylcellulose, hydroxyethylcellulose, hydroxypropylcellulose, sodium carboxymethylcellulose), polyvinyl derivatives such as polyvinyl alcohol, polyvinylpyrrolidone, polyvinyl acetate, alginates, polyacrylates (for example, Carbopol), xanthans, and pectins.

As aqueous phase A it is possible to use water, aqueous solutions or mixtures of water with water-miscible liquids such as glycerol or polyethylene glycol. Further, additional components for the aqueous phase are, for example, mannose, glucose, fructose, xylose, trehalose, mannitol, sorbitol, xylitol or other polyols such as polyethylene glycol and also electrolytes such as sodium chloride. These additional components can be used in an amount of 1% to 60% by weight, for example, 1% to 30% by weight, based on the aqueous phase A.

If desired it is possible, furthermore, to use viscosity enhancers or charge carriers as are described in EP-B-0 605 497. Thickeners which can be used include, for example, polysaccharides, polyalkyl acrylates, polyalkyl cyanoacrylates, polyalkylvinyl-pyrrolidones, acrylic polymers, polylactic acids or polylactides.

As emulsifiers which form lyotropic LC structures or lamellar structures it is possible to use natural or synthetic products. The use of surfactant mixtures is a further possibility. Examples of suitable emulsifiers are the physiological bile salts such as sodium cholate, sodium dehydrocholate, sodium deoxycholate, sodium glycocholate, and sodium taurocholate. Animal and plant phospholipids such as lecithins together with their hydrogenated forms, and also polypeptides such as gelatin, with their modified forms, may also be used.

Suitable synthetic surface-active substances are the salts of sulfosuccinic esters, polyoxyethylene acid betaine esters, acid betaine esters and sorbitan ethers, polyoxyethylene fatty alcohol ethers, polyoxyethylenestearic esters, and corresponding mixture condensates of polyoxyethylene-methpolyoxypropylene ethers, ethoxylated saturated glycerides, partial fatty acid glycerides and polyglycides. Examples of suitable surfactants are Biobase® EP and Ceralution® H.

Examples of suitable emulsifiers are, additionally, glyceryl esters, polyglyceryl esters, sorbitan esters, sorbitol esters, fatty alcohols, propylene glycol esters, alkylglucositol esters, sugar esters, lecithin, silicone copolymers, lanolin, and mixtures or derivatives thereof. Glyceryl esters, polyglyceryl esters, alkoxylates and fatty alcohols, and also isoalcohols, may be derived, for example, from castor fatty acid, 12-hydroxystearic acid, isostearic acid, oleic acid, linoleic acid, linolenic acid, stearic acid, myristic acid, lauric acid, and capric acid.

Besides the stated esters it is also possible for succinates, amides or ethanolamides of the fatty acids to be present. Particularly suitable fatty acid alkoxylates are the ethoxylates, propoxylates or mixed ethoxylates/propoxylates. A further possibility is to use silicone surfactants such as silicone copolyols and silicone betaines.

In accordance with the invention it is preferred to use emulsifier systems whose mixtures of coemulsifiers (gel network formers such as fatty alcohols, fatty acids, sorbitan esters, etc) and specific surfactants form myelin structures at the interface with water. Suitable surfactants include, for example, polyglyceryl-10 tricaprylate, polyglyceryl-10 trilaurate, polyglyceryl-2 oleate, sodium lauroyl lactylate, sodium cocoyl lactylate and glyceryl cocoate citrate lactylate.

It is also possible with preference to use balanced complex emulsifiers.

The optimum ratio of hydrophilic surfactant to coemulsifier for producing MSSN is preferably higher than the optimum ratio for the formation of a gel network.

Waxes/polymers/lipids and emulsifiers are used preferably in a weight ratio of 50:1 to 2:1, preferably 15:1 to 30:1.

The active pharmaceutical, cosmetic and/or food technology compounds are used, based on phase B, in an amount of preferably 0.1% to 70% by weight, more preferably 1% to 10% by weight.

Listed below by way of example are active pharmaceutical compounds, which may be used, for example, in free form, as the salt, or as esters or ethers:

Analgesics/anti-inflammatories, such as morphine, codeine, piritramide, fentanyl and fentanyl derivatives, levomethadone, tramadol, diclofenac, ibuprofen, indometacin, naproxen, piroxicam, penicillamine; antiallergics, such as pheniramine, dimetindene, terfenadine, astemizole, loratadine, doxylamine, meclozine, bamipine, clemastine; antibiotics/chemotherapeutics, such as polypeptide antibiotics such as colistin, polymyxin B, teicoplanin, vancomycin; antimalarials such as quinine, halofantrin, mefloquine, chloroquine, virostatics such as ganciclovir, foscarnet, zidovudine, aciclovir and others such as dapsone, fosfomycin, fusafungine, trimetoprim; antiepileptics, such as phenyloin, mesuximide, ethosuximide, primidone, phenobarbital, valproic acid, carbamazepine, clonazepam; antimycotics, such as internals: nystatin, natamycin, amphotericin B, flucytosine, miconazole, fluconazole, itraconazole; and externals: clotrimazole, econazole, tioconazole, fenticonazole, bifonazole, oxiconazole, ketoconazole, isoconazole, tolnaftate; corticoids (internals), such as aldosterone, fludrocortisone, betamethasone, dexamethasone, triamcinolone, fluocortolone, hydroxycortisone, prednisolone, prednylidene, cloprednol, methylprednisolone; dermatologic agents, such as antibiotics: tetracycline, erythromycin, neomycin, gentamycin, clindamycin, framycetin, tyrothricin, chlortetracycline, mupirocin, fusidic acid; virostatics as above, and also: podophyllotoxin, vidarabine, tromantadine; corticoids as above, and also: amcinonide, fluprednidene, aclometasone, clobetasol, diflorasone, halcinonid, fluocinolone, clocortolone, flumethasone, difluocortolone, fludroxycortide, halometasone, desoximetasone. fluocinolide, fluocortin butyl, fluprednidene, prednicarbate, desonide; diagnostic agents, such as radioactive isotopes such as Te99m, In111 or I131, covalently bonded to lipids or lipoids or other molecules or in complexes, highly substituted iodine-containing compounds such as, for example, lipids; hemostyptics, such as blood coagulation factors VIII, IX; hypnotics, sedatives, such as cyclobarbital, pentobarbital, phenobarbital, methaqualone, benzodiazepines (flurazepam, midazolam, netrazepam, lormetazepam, flunitrazepam, trazolam, brotizolam, temazepam, loprazolam); hypophyseal hormones, hypothalamus hormones, regulatory peptides and their inhibitors, such as corticotrophin, tetracosactide, chorionic gonadotropin, urofollitropin, urogonadotropin, somatropin, metergoline, bromocriptine, terlipressin, desmopressin, oxytocin, argipressin, ornipressin, leuprorelin, triptorelin, gonadorelin, buserelin, nafarelin, goselerin, somatostatin; immunotherapeutics and cytokines, such as dimepranol 4-acetamidobenzoate, thymopentin, α-interferon, β-interferon, filgrastim, interleukins, azathioprine, ciclosporin; local anesthetics, such as internals: butanilicaine, mepivacaine, bupivacaine, etidocaine, lidocaine, articaine, prilocalne; and externals: propitocaine, oxybuprocaine, etracaine, benzocaine; antimigraine agents, such as proxibarbal, lisuride, methysergide, dihydroergotamine, clonidine, ergotamine, pizotifen; narcotics, such as methohexital, propofol, etomidate, ketamine, alfentanil, thiopental, droperidol, fentanyl; parathyroid hormones, calcium metabolism regulators, such as dihydrotachysterol, calcitonin, clodronic acid, etidronic acid; ophthalmic agents, such as atropine, cyclodrine, cyclopentolate, homatropine, tropicamide, scopolamine, pholedrine, edoxudine, idoxuridine, tromantadine, aciclovir, acetazolamide, diclofenamid, carteolol, timolol, metipranolol, betaxolol, pindolol, befunolol, bupranolol, levobunolol, carbachol, pilocarpine, clonidine, neostigmine; psychopharmaceuticals, such as benzodiazepines (lorazepam, diazepam), clomethiazole; thyroid gland therapeutic agents, such as 1-thyroxine, carbimazole, thiamazole, propylthiouracil; sera, immunoglobulins, vaccines, such as immunoglobulins generally and specifically such as hepatitis types, German measles, cytomegalovirus, rabies; TBE, varicella zoster, tetanus, rhesus factors, immune sera such as botulism antitoxin, diphtheria, gas gangrene, snake poison, scorpion venom, vaccines, such as influenza, tuberculosis, cholera, diphtheria, hepatitis types, TBE, German measles, Haemophilus influenzae, measles, Neisseria, mumps, poliomyelitis, tetanus, rabies, typhus; sex hormones and their inhibitors, such as anabolics, androgens, antiandrogens, gestagens, estrogens, antiestrogens (tamoxifen etc.); cytostatics and metastase inhibitors, such as alkylating agents such as nimustine, melphalan, carmustine, lomustine, cyclophosphamide, ifosfamide, trofosfamide, chlorambucil, busulfan, treosulfan, prednimustine, thiotepa, antimetabolites such as cytarabine, fluorouracil, methotrexate, mercaptopurine, tioguanine, alkaloids such as vinblastin, vincristin, vindesin; antibiotics such as aclarubicin, bleomycin, dactinomycin, daunorubicin, epirubicin, idarubicin, mitomycin and plicamycin, complexes of transition group elements (for example Ti, Zr, V, Nb, Ta, Mo, W, Pt) such as carboplatin, cisplatin, and metallocene compounds such as titanocene dichloride, amsacrine, dacarbazine, estramustine, etoposide, hydroxycarbamid, mitoxantrone, procarbazine and temiposide, alkylamido phospholipids (described in J. M. Zeidler, F. Emling, W. Zimmermann and H. J. Roth, Archiv der Pharmazie, 324 (1991), 687), and ether lipids such as hexadecylphosphocholine, ilmofosine and analogs, described in R. Zeisig, D. Arndt and H. Brachwitz, Pharmazie 45 (1990), 809 to 818.

Examples of further suitable active compounds include diclofenac, ibuprofen, acetylsalicylic acid, salicylic acid, erythromycin, ketoprofen, cortisone, and glucocorticoids.

Additionally suitable are active cosmetic compounds, which in particular are sensitive to oxidation or hydrolysis, such as polyphenols, for example. Mention may be made here of catechins (such as epicatechin, epicatechin 3-gallate, epigallocatechin, epigallocatechin 3-gallate), flavonoids (such as luteolin, apigenin, rutin, quercitin, fisetin, kaempherol, rhamnetin), isoflavones (such as genistein, daidzein, glycitein, prunetin), coumarins (such as daphnetin, umbelliferone), emodin, resveratrol, and oregonin.

Suitable vitamins include retinol, tocopherol, ascorbic acid, riboflavin, and pyridoxine.

Suitability is possessed, furthermore, by whole extracts from plants that include above molecules or classes of molecule.

According to one embodiment of the invention the active compounds are sunscreen agents. They may be present in the form of organic sunscreen agents at room temperature (25° C.) in liquid or solid form. Suitable sunscreen agents (UV filters) are, for example, compounds based on benzophenone, diphenyl cyanoacrylate or p-aminobenzoic acid. Specific examples are (INCI or CTFA names) Benzophenone-3, Benzophenone-4, Benzophenone-2, Benzophenone-6, Benzophenone-9, Benzophenone-1, Benzophenone-11, Etocrylene, Octocrylene, PEG-25 PABA, Phenylbenzimidazole Sulfonic Acid, Ethylhexyl Methoxycinnamate, Ethylhexyl Dimethyl PABA, 4-Methylbenzylidene Camphor, Butyl Methoxy-dibenzoylmethane, Ethylhexyl Salicylate, Homosalate, and Methylene-Bis-Benzotriazolyl Tetramethylbutylphenol (2,2′-methylene-bis{6-(2H-benzotriazol-2-yl)-4-(1,1,3,3-tetramethylbutyl)phenol}, 2-hydroxy-4-methoxy-benzophenone-5-sulfonic acid, and 2,4,6-trianilino-p-(carbo-2′-ethylhexyl-1′-oxy)-1,3,5-triazine.

Further organic sunscreen agents are octyltriazones, avobenzones, octyl methoxycinnamates, octyl salicylates, benzotriazoles, and triazines.

According to a further embodiment of the invention, active compounds used are active antidandruff agents, such as are customarily present in cosmetic or pharmaceutical formulations. One example thereof is Piroctone Olamine (1-hydroxy-4-methyl-6-(2,4,4-dimethylpentyl)-2-(1H)-pyridone, preferably in combination with 2-aminoethanol (1:1)). Further suitable agents for treating dandruff are known to the skilled worker.

Suitable active compounds further include, for example, all oxidation-sensitive active compounds such as tocopherol.

According to one further embodiment of the invention, organic dyes are used as or in lieu of active compounds.

Further suitable active compounds are insect repellents and, in the field of food technology, aromas and flavors. Suitable aromas and flavors are known to the skilled worker.

It is also possible, furthermore, to incorporate pigmentary inorganic solids such as TiO₂ and ZnO into the active compound vehicles.

By means of the emulsifiers it is possible to form a unilamellar or multilamellar system or a lyotropic liquid-crystalline mixed phase.

The average diameter of the active compound particles is preferably 50 to 1000 nm, more preferably 100 to 500 nm.

The invention also provides an aqueous active compound vehicle dispersion obtainable in accordance with the above method.

The invention provides, furthermore, a method for producing a multiple dispersion by mixing a dispersion produced as described above with a further polyol phase or oil phase. The invention provides as well a multiple dispersion thus produced. Multiple emulsions are described for example in DE-A-43 41 113.

The invention provides, furthermore, drugs, cosmetics or food additives comprising an above-described dispersion or multiple dispersion.

Further ingredients of the aqueous active compound vehicle dispersions produced in accordance with the invention are described in EP-B-0 605 497, EP-B-0 167 825, and U.S. Pat. No. 5,885,486. Particularly with regard to suitable stabilizing substances and charge stabilizers, attention is drawn to EP-B-0 605 497.

According to one embodiment of the invention the active compound vehicle dispersions are produced with the use of halogenated organic solvents excluded.

The drugs can be administered intravenously, intramuscularly, intraarticularly, intracavitally, subcutaneously, intradermally, enterally, pulmonarily, and also by topical or ophthalmological application.

The invention is elucidated in more detail by the examples below.

### EXAMPLES

In the examples below the following compounds were employed:

| Trade name | Manufacturer | CTFA/INCI |
|---|---|---|
| Pationic ® 138 C | RITA | Sodium lauroyl lactylate |
| Pluronic ® F | BASF | Poloxamer 237 |
| Tween ® | Uniqema | Polysorbate 20 [illegible] |
| Keltrol ® | NutraSweet | Xanthum gum |
| Tylose ® C 300 P 2 | Clariant | Sodium carboxymethylcellulose |
| Softisan ® 142 | Hüls AG | Hydrogenated cocoglycerides |
| Cetiol ® MM | Cognis | Myristyl myristate |
| Cutina ® CP | Cognis | Cetyl palmitate |
| Tocopherol ® | Roche | Tocopherol |
| Ceralution ® H | Sasol | Glyceryl stearate, C18-C22 |
| | | alcohol, C20-C22 alcohol, |
| | | sodium dicocoylethylene |
| Phenonip ® | Nipa | Phenoxyethanol, methylparaben, |
| | | ethylparaben, butylparaben, |
| | | propylparaben, iso |
| Pationic ® SSI | RITA | Sodium stearoyl lactylate |
| Lanette ® E | Cognis | Sodium cetearyl sulfate |
| Sistema ® L 70.C | Sisterna | Sucrose laurate, water, |
| | | ethanol |
| Biobase ® EP | Tri-K | Glyceryl stearate, cetearyl |
| | | alcohol, sodium stearoyl |
| | | lactylate, lecithin |
| Gummi Arabicum | Merck | Gum arabic |
| Guar HV 7000 CPS | B + V S.R.L. | |
| Pectin USP | Dansico | Pectin |
| Bienenwachs | Paramelt | Beeswax |
| Candelillawachs | Strahl + Pitch | Candelilla wax |
| Ceralution ® F | Sasol | Sodium dicocoylethylene- |
| | | diamine PEG-15 sulfate, |
| | | sodium lauroyl lactylate |

The aqueous active compound vehicle dispersion was produced by separately heating phases A and B, described below, to 60° C. Phase B was then stirred into phase A, and using a Braun kitchen mixer (maximum power 350 W) with an agitating-blade diameter of 50 mm the mixture was homogenized until the droplet size was below 350 nm. Then, at room temperature, phase C, likewise at room temperature, was added to the hot emulsion. Agitation was again carried out with a Braun kitchen mixer.

The last three lines of the tables which follow specify the average particle diameter, the weight fraction of particles having a diameter of less than 1 μm, and the specific surface area (cm²/cm³). The compositions of the individual phases and the stated parameters are evident from the following tables.

Example

| | 1 | 2 | 3 | 4 | 5 | 6 |
|---|---|---|---|---|---|---|
| Phase A | | | | | | |
| demin. water | 10 | 8 | 8 | 8 | 8 | 8 |
| Pationic 138 A | 0.75 | 0.75 | 0.75 | 0.75 | 0.75 | |
| Pluronic F 127 | | | | | | 0.75 |
| Tween 20 | | | | | 0.5 | 0.5 |
| Keltrol | 0.35 | 0.35 | | 0.35 | 0.35 | 0.35 |
| Tylose C 300 P 2 | | | 0.354 | | | |
| Phase B | | | | | | |
| Softisan 142 | 20 | 20 | 20 | | 20 | 20 |
| Cetiol MM | | | | 20 | | |
| Cutina CP | | | | | | |
| Tocopherol | 1 | 1 | 1 | 1 | 1 | 1 |
| Ceralution H | 1.75 | 1.75 | 1.75 | 1.75 | 1.25 | 1.25 |
| Phase C | | | | | | |
| demin. water | 65.55 | 67.55 | 67.55 | 67.55 | 67.55 | 67.55 |
| Phenonip | 0.6 | 0.6 | 0.6 | 0.6 | 0.6 | 0.6 |
| | 100 | 100 | 100 | 100 | 100 | 100 |
| PSA | | | | | | |
| median [μm] | 0.40 | 0.39 | 0.39 | 0.39 | 0.35 | 0.44 |
| <1 μm [%] | 99.8 | 99.7 | 99.8 | 99.8 | 100.0 | 97.7 |
| cm²/cm³ | 145 062 | 150 707 | 151 944 | 150 731 | 168 821 | 128 440 |

Example

| | 7 | 8 | 9 | 10 | 11 | 12 |
|---|---|---|---|---|---|---|
| Phase A | | | | | | |
| demin. water | 8 | 8 | 8 | 8 | 8 | 8 |
| Pationic 138 A | 0.75 | 0.35 | 0.35 | 0.5 | 0.5 | 0.25 |
| Pluronic F 127 | 0.5 | | | | | |
| Tween 20 | | 0.3 | 0.75 | 0.75 | 0.75 | 0.38 |
| Keltrol | 0.35 | 0.35 | 0.35 | 0.35 | 0.35 | 0.35 |
| Tylose C 300 P 2 | | | | | | |
| Phase B | | | | | | |
| Softisan 142 | 20 | 20 | 20 | 20 | | |
| Cetiol MM | | | | | | |
| Cutina CP | | | | | 20 | 20 |
| Tocopherol | 1 | 1 | 1 | 1 | 1 | 1 |
| Ceralution H | 1.25 | 1.85 | 1.4 | 1.25 | 1.25 | 0.63 |
| Phase C | | | | | | |
| demin. water | 67.55 | 67.55 | 67.55 | 67.55 | 67.55 | 68.79 |
| Phenonip | 0.6 | 0.6 | 0.6 | 0.6 | 0.6 | 0.6 |
| | 100 | 100 | 100 | 100 | 100 | 100 |
| PSA | | | | | | |
| median [μm] | 0.40 | 0.41 | 0.36 | 0.35 | 0.34 | 0.50 |
| <1 μm [%] | 99.7 | 99.7 | 100.0 | 100.0 | 100.0 | 97.0 |
| cm²/cm³ | 148 410 | 144 299 | 164 857 | 171 685 | 174 428 | 116 783 |

| | Example | | | | | | | |
|---|---|---|---|---|---|---|---|---|
| | 13 | 14 | 15 | 16 | 17 | 18 | 19 | 20 |
| Phase A | | | | | | | | |
| demin. water | 8 | 8 | 8 | 8 | 8 | 8 | 8 | 8 |
| Pationic 138 A | 0.75 | 0.5 | 0.5 | 0.5 | 0.25 | 0.5 | 0.5 | 0.25 |
| Lanette E | | | 0.5 | | | | | |
| Sisternal L 70 C | | | | 0.5 | | | | |
| Tween 20 | | 0.5 | | | 0.75 | 0.75 | 0.75 | 0.38 |
| Keltrol | 0.35 | 0.35 | 0.35 | 0.35 | 0.35 | 0.35 | 0.35 | 0.35 |
| Phase B | | | | | | | | |
| Softisan 142 | 20 | 20 | 20 | 20 | 20 | 20 | | |
| Cetiol MM | | | | | | | 20 | 20 |
| Tocopherol | 1 | 1 | 1 | 1 | 1 | 1 | 1 | 1 |
| Biobase EP | 1.75 | 1.5 | 1.5 | 1.5 | 1.5 | 1.25 | 1.25 | 0.63 |
| Phase C | | | | | | | | |
| demin. water | 67.55 | 67.55 | 67.55 | 67.55 | 67.55 | 67.55 | 67.55 | 68.79 |
| Phenonip | 0.6 | 0.6 | 0.6 | 0.6 | 0.6 | 0.6 | 0.6 | 0.6 |
| | 100 | 100 | 100 | 100 | 100 | 100 | 100 | 100 |
| PSA | | | | | | | | |
| median [μm] | 0.51 | 0.35 | 0.39 | 0.43 | 0.35 | 0.34 | 0.34 | 0.5 |
| <1 μm [%] | 94.0 | 100.0 | 99.6 | 99.5 | 100.00 | 100.00 | 100.00 | 97.1 |
| cm²/cm³ | 109 422 | 172 693 | 148 338 | 137 636 | 169 830 | 172 249 | 173 520 | 115 983 |

Example

| | 21 | 22 | 23 | 24 | 25 | 26 |
|---|---|---|---|---|---|---|
| Phase A | | | | | | |
| demin. water | 8 | 8 | 8 | 8 | 8 | 8 |
| Pationic 138 A | 0.5 | 0.5 | 0.5 | 0.5 | 0.15 | 0.5 |
| Tween 20 | 0.75 | 0.75 | 0.75 | 0.75 | 0.75 | 0.75 |
| Keltrol | 0.35 | 0.5 | 0.35 | 0.35 | | |
| Guar HV 7000 CPS | | | | | 0.35 | |
| Pectin USP | | | | | | 0.35 |
| Phase B | | | | | | |
| Cetiol MM | 20 | 20 | 18 | | 20 | 20 |
| Bees Wax Care 144 | | | | 20 | | |
| Tocopherol | 1 | 1 | 3 | 1 | 1 | 1 |
| Ceralution H | 1.25 | 1.25 | 1.25 | 1.25 | 1.25 | 1.25 |
| Phase C | | | | | | |
| demin. water | 67.55 | 67.4 | 67.55 | 67.55 | 67.55 | 67.55 |
| Phenonip | 0.6 | 0.6 | 0.6 | 0.6 | 0.6 | 0.6 |
| PSA | | | | | | |
| median [μm] | 0.34 | 0.33 | 0.35 | 0.38 | 0.43 | 0.41 |
| <1 μm [%] | 100.0 | 100 | 100 | 98.2 | 98.8 | 98.4 |
| cm²/cm³ | 176 844 | 181 470 | 168 608 | 137 601 | 135 134 | 131 861 |

1-15. (canceled)

16. A method for producing an aqueous vehicle dispersion in which there are solid active compound vehicle particles which are based on wax, polymer or lipid, have an average diameter in the range from 10 to 10 000 nm, and comprise at least one active pharmaceutical, cosmetic and/or food technology compound, fragrance or flavor, by
a) mixing the active compound with the wax-, polymer- or lipid-based active compound vehicle and at least one emulsifier which leads in stage b) to the formation of a lyotropic liquid-crystalline mixed phase, at a temperature above the melting or softening point of the active compound vehicle, to form a phase B,
b) mechanically mixing the phase B with an aqueous phase A, which may comprise an emulsifier, at a temperature above the melting or softening point of the active compound vehicle, the weight ratio of phase B to phase A being 1:5 to 5:1, without high-pressure homogenization, to form a—preferably gellike—lyotropic liquid-crystalline mixed phase,
c) diluting the mixed phase with an aqueous phase, which may comprise an emulsifier, at an aqueous-phase temperature which is below the melting or softening point of the active compound vehicle, with stirring and without high-pressure homogenization, to a desired final concentration of the dispersion.

17. The method of claim 16, characterized in that the mechanical mixing in stage b) and the stirring in stage c) take place with agitators which have a peripheral speed in the range from 1 to 20 m/s.

18. The method of claim 17, characterized in that the shearing action of the agitator corresponds to the shearing action of a household kitchen mixer.

19. The method of claim 16, characterized in that the weight ratio of phase B to phase A in stage b) is 1:2 to 2:1.

20. The method of claim 16, characterized in that the active compound vehicle particles are based on diglycerides, triglycerides, fatty alcohols, their esters or ethers, waxes, lipid peptides or mixtures thereof.

21. The method of claim 16, characterized in that the average diameter of the active compound vehicle particles is 50 to 1000 nm.

22. An aqueous active compound vehicle dispersion obtainable by a method as claimed in claim 16.

23. A method for producing a multiple dispersion by mixing a dispersion prepared by a method as claimed in claim 16 with a further polyol phase or oil phase.

24. A multiple dispersion obtainable by a method as claimed in claim 23.

25. A drug, a cosmetic or a food additive comprising a dispersion as claimed in claim 22.

26. Membrane-structured solid nanoparticles having an average diameter in the range from 10 to 10 000 nm which are solid at 25° C. and have a combination of active compound vehicle particles and emulsifiers such as to form membranes which infiltrate the entire nanoparticles so that there are emulsifiers in the interior and on the surface of the Nanoparticles, producible by a method as claimed in claim 16.

27. The nanoparticles of claim 26, characterized in that there are essentially no regions without a membrane structure over the cross section of the nanoparticles.

28. The nanoparticles of claim 26, characterized in that the membranes are formed in a lyotropic liquid-crystalline mixed phase which in the presence of water is self-emulsifying.

29. The nanoparticles of claim 26, characterized in that they are loaded with at least one active pharmaceutical, cosmetic and/or food technology compound in an amount of up to 60% by weight, based on the loaded nanoparticles.

30. The nanoparticles of claim 29, characterized in that they are loaded with sunscreen agents.

31. A drug, a cosmetic or a food additive comprising a multiple dispersion as claimed in claim 24.
